(19) United States
(12) Patent Application Publication
(10) Pub. No.: US 2014/0277220 A1
(43) Pub. Date: Sep. 18, 2014

(54) HEATED GARMENT FOR MEDICAL APPLICATIONS

(71) Applicant: Birch Tree Medical, Inc., Newbury, MA (US)

(72) Inventors: Patrick Brennan, Newbury, MA (US); Anthony K. Iacobucci, West Newbury, MA (US)

(73) Assignee: Birch Tree Medical, Inc., Newbury, MA (US)

(21) Appl. No.: 14/178,824

(22) Filed: Feb. 12, 2014

Related U.S. Application Data
(60) Provisional application No. 61/785,839, filed on Mar. 14, 2013.

(51) Int. Cl. A61F 7/02; A61N 1/36

(52) U.S. Cl. CPC A61F 7/02; A61N 1/36003; A61F 2007/0233; A61F 2007/0094
USPC 607/3; 607/112; 607/100

(57) ABSTRACT

A patient-treatment system and related method provide relief from, and treatment for, muscle spasticity disorders, Willis-Ekbom disease, contracture, sleep onset insomnia, sleep maintenance insomnia, rheumatoid arthritis, and other similar disorders by applying controlled heat, and optionally muscle monitoring/stimulation, to one or more parts of a patient's body.

### RELATED APPLICATION

This application claims the benefit of priority from U.S. Provisional Patent Application No. 61/785,839 entitled “Heated Garment for Medical Applications” that was filed on Mar. 14, 2013, the entirety of which is hereby incorporated herein by reference.

### TECHNICAL FIELD

In various embodiments, the present invention relates to methods and systems for treating muscle spasticity disorders, Willis-Ekbom disease (also known as restless leg syndrome (RLS)), muscle and tendon contracture, sleep onset insomnia, sleep maintenance insomnia, joint stiffness of rheumatoid arthritis, and other similar disorders. More particularly, embodiments of the present invention relate to a heat delivery and muscle stimulation system that can provide relief from those disorders by applying controlled heat to one or more parts of a patient's body.

### BACKGROUND

Spasticity generally results from an injury to the upper motor neuron of the central nervous system, such as after a stroke, a spinal cord injury, a traumatic brain injury, and cerebral palsy. It is one of five factors that contribute to disability in each of these conditions (weakness, contracture, motor control, and habit being the other four). Spasticity both results in, and prevents effective therapy of, the other four contributing factors. Spasticity is life-long and it currently has no cure. Willis-Ekbom disease and insomnia cause sleep deprivation, while contractures (caused by non-neurologic reasons) and arthritis affect independence in activities of daily living also.

### Spasticity

Spasticity is generally defined as velocity-dependent muscle tone due to an injury to the upper motor neuron of the central nervous system, and is typically sensed by an affected individual as involuntary muscle contraction of a part of the body during voluntary movement of that body part. More specifically, the muscles that the individual voluntarily uses, and the muscles that oppose that action, contract in such a way as to make the voluntary movement more difficult. For instance, the hamstring muscles may keep the knee bent, as the individual is attempting to extend the knee while walking As another example, the wrist flexors often contract and work against the muscles that open the hand. Typically, botulinum toxin is injected into those opposing muscles, providing temporary (e.g., 4-6 months) benefit, but also a window of time for more permanent benefit, as discussed below. Spasticity impairs independent function in daily living activities, and thereby creates a dependence on caregivers and mechanical aids. In the case of stroke, spasticity increases the cost of care fourfold compared to stroke without spasticity.

Spasticity is one of the most disabling features of multiple conditions that affect adults and children, including cerebral palsy, stroke, spinal cord injury, traumatic brain injury, central nervous system diseases, and tumors of the nervous system. Spasticity can lead to contracture, which is a gradual abnormal shortening of the muscle. Contracture can typically be prevented and treated with a simple stretching program, however a spastic muscle tightens and contracts when it is stretched, and does not lengthen as a relaxed muscle does when stretched. The lifelong consequences of a stroke and of the other conditions listed above include muscle weakness, poor motor control, and a new, ingrained firing pattern (a “habit”) from the brain to the muscle that is less advantageous to function than the firing pattern that existed prior to the injury or disease.

It is very difficult for individuals to re-learn movement patterns when spasticity is present, and much easier for them to re-learn when it is absent. Studies of the spasticity-lowering effects of botulinum toxin show that decreasing spasticity helps to maintain range of motion at a joint, thereby preventing contracture, and decreases the need for muscle-lengthening surgeries after stroke and after cerebral palsy. Moreover, per therapist and patient reports, spasticity treatment makes therapy sessions more effective because, with spasticity lessened, motor re-learning is easier, and strengthening exercises are more effective. In particular, therapy sessions are more efficient with spasticity decreased because, in such a case, the muscle opposite the spastic muscle can be strengthened. Increased strength and length of muscles are long-term benefits that cannot be achieved effectively with spasticity working against the therapy. Alleviating spasticity through heat allows a muscle to be stretched so that contracture is avoided and range of motion improved; to be strengthened so that weakness is avoided; and to be re-trained so that abnormal firing patterns may be prevented or improved.

Often, when a patient who has sustained a stroke attempts to move an extremity, all muscles contract. For instance, a voluntary bicep contraction to flex the elbow is often accompanied by an involuntary, unwanted contraction of the triceps, whose action is to extend the elbow. In this case, the tricep is functioning as an antagonist to elbow flexion. This phenomenon is referred to as co-contraction, which impairs movements and, therefore, activities of daily living. Heat applied to the antagonist decreases its action, and thereby eases movement, improving function. The application of heat to a spastic muscle is generally known to decrease spasticity, and is often used as a temporary measure by physical and occupational therapists in the form of heat packs to allow a stretch of the muscle during a therapy session.

In general, spasticity differs from a plain muscle spasm in that a muscle spasm is a short-term, intermittent phenomenon not associated with central nervous system injury. For example, any typical person might experience a muscle spasm while swimming or during maximal athletic exertion as commonly witnessed on televised athletic events, prompting massage by a trainer. A muscle spasm does inhibit lengthening of the muscle, similar to spasticity, but only during the actual spasm. In contrast, spasticity occurs during the majority of a person's movements on a daily basis, causes daily disability, and will usually persist for that person's entire lifetime, as a result of their stroke, spinal cord injury, traumatic brain injury, or cerebral palsy (among many other diseases and injuries at and above the level of the upper motor neuron of the central nervous system). Spasticity can be consistently demonstrated by rapidly moving a patient's joint through its range of motion at varying speeds, and is clinical evidence of central nervous system disease—again an injury to the upper motor neuron. A muscle spasm is not a result of an upper motor neuron injury, but is rather due to pain stimuli to the lower motor neuron.

Current treatments for spasticity (and their drawbacks) include:
a. Physical and occupational therapy techniques including stretching, hot packs, cold packs, and hydrotherapy: these treatments, and the relief experienced, are temporary; for example, a hot pack may be applied to a spastic muscle of a patient for a few minutes during a therapy session to temporarily relieve spasticity so that the muscle can be stretched; as another example, a patient may be placed in a heated pool (typically 94-96 degrees Fahrenheit) for a therapy session lasting 30 minutes or so, which has been commonly noted by therapists to result in decreased spasticity for several hours;
b. Oral medications: baclofen is often very effective for spasticity associated with spinal cord injury; however, for other indications, oral medications are poorly effective, and can produce side effects such as sedation (tizanidine) and toxicity to the liver or kidneys; parents are understandably hesitant to administer these to their children;
c. Medications injected into muscles: while botulinum toxin and phenol are effective, they are also expensive, painful at the time of administration (often requiring general anesthesia), temporary (lasting at most 6 months in the case of botulinum toxin, and up to one year with phenol), often repeated as spasticity returns, susceptible to initial dosing that may be too low or too high, and can at high doses have systemic effects;
d. Medication delivered directly to the spinal cord: an intrathecal baclofen pump (ITB) may be surgically implanted into a patient's abdomen, with a catheter running under the skin, to deliver liquid baclofen directly to the nerves of the spinal cord that influence spasticity in the leg muscles; the ITB is very effective against the spasticity, it improves walking patterns, its benefits can be long-lasting, and it is adjustable; however, potential drawbacks include failure of the pump and acute baclofen withdrawal (which, if untreated, can be fatal), infection, damage to surrounding tissues, blockage or disconnection of the catheter, impaired functioning of the bowel and bladder, and tissue damage around the pump; and
e. Surgical procedure (rhizotomy): a surgical procedure in which the spinal cord is exposed, the nerves influencing spasticity are identified, and some percent of those nerves are cut can be very effective and long lasting in its spasticity-lowering effects, but it is not used in adult spasticity; surgery only helps with leg spasticity in most cases, is unable to be adjusted, can impair bowel and bladder function, may lead to scoliosis, and may unmask dramatic weakness requiring intensive therapy.

### Willis-Ekbom Disease

Willis-Ekbom disease, also known as Restless Leg Syndrome (RLS), is a common disorder that affects children and adults in approximately 10% of the population. Uncomfortable sensations in the legs, beginning in the evening, can significantly interfere with sleep and negatively impact quality of life. This disease is lifelong; currently has no cure except in cases of low iron as the sole cause; and is thought to have both genetic and brain metabolism related (iron, dopamine) abnormalities as its causes. Willis-Ekbom disease often spreads with time, from affecting only the legs, to also affecting the muscles of the arms and torso. Symptoms can also worsen in duration, with Willis-Ekbom disease occurring during all waking hours in severe cases. Patients describe an intensely uncomfortable sensation that impairs and often prevents sleep, temporarily eased by hot showers that may allow for two to three hours of sleep. The consequences of sleep deprivation for the individual, and for society, are enormous when all causes are included (obstructive sleep apnea, etc.).

Current FDA-approved treatments for Willis-Ekbom disease include medications that increase dopamine levels in the brain (e.g., Mirapex and Requip). Common adverse side-effects of these medications include nausea, headache, dry mouth, and augmentation (i.e., the onset of symptoms earlier in the day and increased severity of the symptoms), and a significant number of patients describe side-effects from dopaminergic medications. Many patients remain on these medications and tolerate side-effects, only because the effects of long-term sleep deprivation are worse. Augmentation can occur in approximately 50-85% of patients receiving Levodopa. Ropinirole (Mirapex) produces nausea in approximately 38% of patients. A new, conservative, potential first-line treatment would be welcomed by patients and physicians.

In a survey by Dr. Julienne Winkelmenn on the topic, a majority of patients with Willis-Ekbom disease stated that the application of heat to the affected body part significantly relieved their symptoms. Dr. John Winkelman of Harvard Medical School, a leading authority on Willis-Ekbom disease, states that his patients report heat as very helpful, and the only way many patients could sleep, prior to the dopamine-based medications.

Approximately 82% of patients suffering from Willis-Ekbom disease have been reported to use temperature change (hot or cold baths) to relieve symptoms. Patients who benefit from heat, usually in the form of a bath, describe the relief as immediate, but temporary as the symptoms return as the heating effect of the bath wears off two to three hours after the patient has left the bath. Patients who gain significant relief from heat may not need medication, or may require a lower dose, and would potentially avoid the side-effects of the medication.

### Contracture, Rheumatoid Arthritis, Sleep Onset Insomnia, and Sleep Maintenance Insomnia

A “contracture” is a muscle that has lost range of motion (e.g., a person's elbow will not go straight or will not fully extend, even while asleep or under anesthesia, since the muscle contains less muscle cells in its entire length than normal). This can occur due to prolonged periods in a flexed position, or due to weakness and/or spasticity. Contracture occurs after the above-described neurologic diseases, but also for non-neurologic reasons (e.g., trauma, electrolyte abnormalities, etc.).

Effective stretching of a relaxed muscle produces increased muscle cell division. Current treatments for contracture (i.e., a physically shortened muscle) include physical therapy, botulinum toxin when spastic, and lengthening surgeries in which the tendons and/or muscles are partially cut and sometimes forcefully stretched during surgery, then immobilized with casting. It is thought that heat's relaxing effect on muscle allows a prolonged stretch, stimulating muscle cell division at the “growth plate” of the muscle (i.e., where the muscle and tendon join). It is absolutely required that the muscle be relaxed, in order for the stretch to have its effect.

Rheumatoid arthritis is a lifelong, currently incurable disease that damages the joints of many parts of the body, including the hands, leading to pain and disability. In treating rheumatoid arthritis, therapists commonly provide heat to a patient's joints, for example by dipping the patient's hands into warm liquid wax, consistently providing significant short-term relief. This is a temporary intervention that is limited to the patient's hands. Other treatments for rheumatoid arthritis include immune-compromising medications (“DMARDS”) that, while often effective, can cause dangerous side effects, such as an increased risk of cancer and gastrointestinal bleeding.

Current treatments for sleep onset insomnia and sleep maintenance insomnia include medications, which risk side effects such as sedation, hangover sleepiness, and addiction.

Accordingly, needs exist for improved systems and methods of treating spasticity, Willis-Ekbom disease, contracture, sleep onset insomnia, sleep maintenance insomnia, and rheumatoid arthritis.

### SUMMARY OF THE INVENTION

In various embodiments, the present invention features a wearable garment, set of garments or other heat delivery system that provide(s) heat to one or more parts of a patient's body, such as spastic extremities, areas of the body that exhibit the symptoms of Willis-Ekbom disease or contracture, and/or muscles that are stiff as a result of a central nervous system injury. For example, in a first aspect, the invention generally discloses a patient-treatment system that includes a wearable garment, e.g., an insulating material that promotes retention of the heat; at least one heating element, coupled to the garment, for applying heat, e.g., at far infrared wavelengths, to a body portion of a wearer of the garment to elicit a response that diminishes symptoms of a condition suffered by the wearer, e.g., spasticity, Willis-Ekbom disease, contracture, sleep onset insomnia, and/or sleep maintenance insomnia; and wearer-controllable circuitry for adjusting the heat output by the heating element. Advantageously, the system allows the wearer to self-treat the condition, the circuitry being further configured to prompt the wearer to accept an automatic scheduling of a therapeutic regimen based on information learned from instructions input by the wearer; and to permit the wearer of the garment to adjust a temperature of the heat output by the heating element.

In some embodiments, the garment includes inner and outer layers and, in some variations of those embodiments, the heating element is positioned between the inner and outer layers.

In still other embodiments, the system may further include one or more of: an electrode coupled to the garment for providing electrical stimulation to the body portion of the wearer of the garment; a lead coupled to the garment for monitoring muscle activity in the body portion of the wearer of the garment; a thermal sensor coupled to the garment for monitoring a skin temperature of the wearer of the garment; a housing that houses at least a portion of the circuitry; a battery housed by the housing; and computer memory in electrical communication with the circuitry. In a variation of these embodiments, the housing is attachable to at least one of a waistline of the wearable garment, a waistline of another item of clothing worn by the wearer of the garment, and a belt worn by the wearer of the garment.

In other variations, a computer memory stores at least one of: the instructions input by the wearer of the garment and data recorded from the wearer's body. Advantageously, the circuitry is remotely controllable by a handheld device employed by the wearer of the garment. Furthermore, the circuitry permits the wearer of the garment to control a first heating element independently from a second heating element. Moreover, the computer memory is remotely interrogable by a clinician.

In a second aspect, the invention generally discloses a method of treating a patient. In some embodiments, the method includes the step of: applying heat to a body portion of the patient via at least one heating element coupled to a garment worn by the patient, the heat eliciting a response that diminishes symptoms of a condition suffered by the patient, the condition selected from the group consisting of spasticity, Willis-Ekbom disease, contracture, sleep onset insomnia, and sleep maintenance insomnia. In other embodiments, the method may further include at least one of: adjusting the applied heat in response to instructions input by the patient while self-treating the condition; prompting the patient to accept an automatic scheduling of a therapeutic regimen based on information learned from the instructions input by the patient; electrically stimulating the body portion of the patient; monitoring muscle activity in the body portion of the patient; monitoring a skin temperature of the patient; receiving, at a receiver associated with the garment, a wireless signal comprising instructions for controlling the application of the heat; storing, in computer memory associated with the garment, at least one of i) instructions input by the patient and ii) data recorded from the patient's body; receiving, at a receiver associated with the garment, a wireless signal comprising instructions to interrogate computer memory associated with the garment; adjusting a temperature of the applied heat in response to instructions input by the patient; and controlling a first heating element independently from a second heating element.

In some variations of some of these embodiments, the garment includes inner and outer layers and/or an insulating material that promotes retention of the heat. In other variations of some of these embodiments, the heating element is positioned between the inner and outer layers. Optionally, the heat may be applied at far infrared wavelengths.

Alternatively, or in addition, the heat may be provided to the patient's body diffusely to simulate a warm bath for the treatment of sleep onset insomnia and/or sleep maintenance insomnia, or to specific joints of the patient that are affected by rheumatoid arthritis. Using the system described herein, various levels of heat and/or muscle stimulation/monitoring can be applied continuously or intermittently throughout the day or night as needed. For example, muscle monitoring with electromyography (EMG) leads embedded in the wearable garment aids in diagnosing and localizing the presence and severity of Willis-Ekbom disease (which is often confirmed as a diagnosis by an overnight sleep study, in which EMG leads detect and count the number of abnormal muscle contractions in the legs during sleep). The same concept is true for spasticity.

Moreover, it has been found, in treating contracture, that if the collagen (i.e., the connective tissue that accumulates in muscle and causes the contracture) is warmed by 2 degrees Fahrenheit, the collagen becomes extensible and lengthens in a lasting manner under the influence of a prolonged stretch, while being heated. Advantages to such a treatment include the avoidance of surgery, minimal discomfort, and no need for immobilization. The rationale for treating both types of insomnia (i.e., sleep onset insomnia and sleep maintenance insomnia) with heat are the common experience, as well as research studies, indicating that a warm bath improves sleep onset and quality, as measured during sleep studies. Embodiments of the present invention provide the benefits of this heat with heated, close-fitting sleepwear, while avoiding the fall risk (especially to the elderly) of bathing. Finally, the rationale for treating rheumatoid arthritis with heat is based on studies indicating that warming the affected joints of patients with rheumatoid arthritis causes the specific gravity (i.e., the “thickness”) of the joint fluid to decrease, making movement easier. By providing day-long, patient-adjustable heat to any and all affected joints, embodiments of the present invention improve these patients' function.

Some of the benefits to the technology described herein (i.e., the wearable, heated garment) over prior art systems/treatments are that it will generally not bring about systemic side effects and there is no possibility of liver or kidney damage. The relief that the wearable, heated garment provides is meant to be long lasting, and the effect is adjustable on a minute-to-minute basis by the wearer. Other advantages to the technology include the fact that it does not cause sedation, it has a relatively low financial cost, it has little to no potential for causing pain (assuming the individual has normal sensation), and there is no known potential for withdrawal symptoms. In addition, in the cases of spasticity and Willis-Ekbom disease, the monitoring qualities of the garment provide valuable data about that patient's movement patterns that can then be used to modify the heat and muscle stimulation features, in order to further improve the patient's function or sleep.

Embodiments of the present invention are non-obvious because some percentage of patients depend on their spasticity to stand, and to transfer from bed to chair, chair to car, etc., and decreasing their spasticity may have an overall negative effect on function and independence. Also, some patients weaken when exposed to heat, which is especially concerning in patients with multiple sclerosis, who often have spasticity. There is also established and published dogma in the rehabilitation field that the preferred temperature change to bring about decreased spasticity is cooling, and not heating.

These and other objects, along with advantages and features of the embodiments of the present invention herein disclosed, will become more apparent through reference to the following description, the accompanying drawings, and the claims. Furthermore, it is to be understood that the features of the various embodiments described herein are not mutually exclusive and can exist in various combinations and permutations.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 illustrates an exemplary embodiment of a garment inner layer in accordance with some embodiments of the present invention;

FIG. 2 illustrates an exemplary embodiment of a garment infrastructure layer in accordance with some embodiments of the present invention;

FIG. 3 illustrates an exemplary embodiment of a garment outer layer in accordance with some embodiments of the present invention;

FIG. 4 illustrates an exemplary embodiment of a central processing management system for the garment in accordance with some embodiments of the present invention; and

FIG. 5 illustrates an exemplary embodiment of a circuit diagram for a central processing management system in accordance with the some embodiments of the present invention.

### DETAILED DESCRIPTION

In broad overview, FIGS. 1-5 show various embodiments of the present invention as they relate to an undergarment 100 or set of undergarments with sewn-in heating elements 15 placed over affected areas of the body and sewn-in electromyographic contacts (EMG leads 11) and/or electrodes 13 placed over certain muscles. The heating elements 15, the electrodes 13, and the EMG leads 11 may be electrically and operationally coupled to a battery supply 48 and CPU 42. The levels of heat and electrical stimulation output by the garment 100 may be programmable and adjustable at any time by a wearer of the garment 100. Moreover, the components included in the specific garment 100 may vary slightly for each disorder to be treated. For example, versions for treating spasticity may include heating elements 15, EMG leads 11 for monitoring, and electrodes 13 for stimulation; versions for treating Willis-Ekbom disease may include heating elements 15 and EMG leads 11 for muscle monitoring; and versions for treating rheumatoid arthritis and/or for treating contracture may include only heating elements 15. In general, the garments 100 are designed to be worn throughout the day and/or night as needed, for example underneath typical clothing during the day and, appropriately, as nightwear.

As mentioned above, heat brings about relaxation of a muscle by decreasing spasticity, relieves the symptoms of Willis-Ekbom disease, improves contracture response to prolonged stretch, decreases insomnia, and loosens stiff joints due to rheumatoid arthritis. In one embodiment, the EMG leads 11 detect, measure, and record muscle contraction, and electrodes 13 can stimulate muscle contraction through battery-supplied electrical stimulation. By detecting muscle contraction, the electronics associated with the garment can localize and grade the severity of disease in some cases (e.g., spasticity, Willis-Ekbom disease). Also, by detecting muscle contraction, compliance with strengthening exercises can be recorded by the CPU 42 and studied. By stimulating muscle contraction, patients become “more aware” of weak muscles, thereby preventing neglect and learned non-use of the muscle. Stimulation directly strengthens weak muscles.

To aid in treating and diagnosing Willis-Ekbom disease, some embodiments of the present invention relate to a heat delivery and muscle monitoring system worn in the evenings and during sleep, but also, as necessary if the disease worsens, worn throughout the day (localizing muscles involved and estimating severity through actigraphy, as in polysomnography).

In general, the wearable, noninvasive, battery-powered, comfortable, patient-controlled device 100 combines heating and electrical monitoring/stimulation components in a panel 47, to prevent, diagnose, treat, and, in some cases, measure various disorders (e.g., muscle spasticity, Willis-Ekbom disease, muscle and tendon contracture, sleep onset insomnia, sleep maintenance insomnia, and joint stiffness of rheumatoid arthritis) and their sequelae. “Prevention” generally refers to preventing the sequelae of these disorders.

A first aspect of the present invention includes a wearable medical garment 100 that is structured and arranged for applying heat and/or muscle stimulation/monitoring to discrete locations on a patient's extremities, trunk muscles, tendons, and/or joints. For illustrative purposes only and not for the purpose of limitation, FIG. 5 shows 16 discrete heating (panel 47) zones (e.g., right arm bicep, right arm tricep, right arm flexor, right arm extensor, left arm bicep, left arm tricep, left arm flexor, left arm extensor, right leg quad, right leg bifemoral, right leg gastrocnemius, right leg shin, left leg quad, left leg bifemoral, left leg gastrocnemius, and left leg shin). More or fewer zones can be incorporated into the garment 100, which may, for example, be in the form of one or more sleeves, or take the form of a jersey 12 and pants 14.

The medical garment 100 may be custom-fit. Alternatively, the medical garment 100 may be self-adjusting and patient-adjustable. In one embodiment, the garment material is similar to thin, snug thermal underwear in appearance, and is adapted to cover any and all areas of the body indicated by specific symptoms. The materials from which the medical garment 100 can be made of include cotton or other comfortable and practical material, or combination of materials in layers, that is/are lightweight, absorbent, anti-microbial or breathable, sweat-wicking, customizable by the patient for design patterns to promote wear by children, and elastic to allow for comfort and growth. Furthermore, the garment may feature wiring connected to a power source 48, such as a battery worn underneath the patient's clothing or suspended from the patient's belt 49. Optionally, the garment may also include a thermoelectric material such as Power Felt, developed by Wake Forest University's Center for Nanotechnology and Molecular Materials, to help generate power from the heat delivered. Thus, the material for the garment may also be constructed of nanostructure fibers that themselves act as rechargeable batteries.

In some embodiments, the garment 100 may include an inner layer 10 (FIG. 1), an outer layer 30 (FIG. 3), and an infrastructure layer 20 (FIG. 2) therebetween. Each layer 10, 20, 30 may be a separately wearable garment or two or more of the layers may be incorporated into a single, wearable garment. For example, in one variation, the infrastructure layer 20 is attached or otherwise integrated into upper 12 and lower portions 14 of the inner layer 10. The infrastructure layer 20 may feature one or more heating panels 47 that include heating elements 15 for applying controllable heat to a bodily portion of a wearer of the garment 100 (i.e., the patient) as well as flexible leads 11 and electrodes 13. The inner layer 10 may be an absorbent, protective lining for use under the heating elements 15 and against the patient's skin. The outer layer 30, which also may include upper 17 and lower 18 portions, may be an insulating layer that is provided over the heating elements 15 to promote the retention of heat. In this way, high-temperature heat may be applied for several minutes and the heat then turned down or off, leaving the garment 100 to contain the heat. Such an approach preserves battery power.

A circuit diagram (FIG. 5) illustrates the device and system. Heat may be applied via heating elements 15 in the panels 47 to the discrete zones so as to elicit a response that diminishes the symptoms and improves the circumstances of a condition suffered by the patient, such as, for example, spasticity, Willis-Ekbom disease, contracture, sleep onset insomnia, sleep maintenance insomnia, and/or rheumatoid arthritis. In some embodiments, each heating panel 47 includes a heating element 15 that applies heat at far infrared wavelengths (i.e., at wavelengths of 15 μm to 1000 μm), which is safe and is not known to cause skin cancer changes, as compared to near infrared heat, which activates the same intermediates as does ultraviolet light (e.g., from the sun) and is known to cause malignant changes. In some embodiments, heat may even be applied at mid-wavelength infrared to long-wavelength infrared wavelengths (i.e., at wavelengths of 3 μm to 15 μm).

In some variations, the infrastructure layer 20 may also feature one or more flexible electrodes 13 for providing electrical stimulation to the patient's weak muscles, one or more flexible EMG leads 11 for monitoring the patient's muscle activity, and/or one or more thermal sensors 17 for monitoring the patient's skin temperature. A thermal shutoff device 41 may be provided to shut-off the heating element 15 in the event that, for example, a short circuit is detected, a ground fault occurs, and/or the sensed temperature exceeds a pre-designated maximum temperature (e.g., 42° C.). Similarly, an electrical shutoff device 43 may be provided to shut-off the leads 11 and/or electrodes 13 in the event that, e.g., a short circuit is detected, a ground fault occurs, and/or a sensed current exceeds a pre-designated maximum current (e.g., 100 mA). The various components of the panel 47 are in electrical communication, through respective thermal 41 and electrical safety shutoffs 43, with patient-controllable circuitry (i.e., a patient-controllable central processing unit (CPU) 42) and random access 44a and flash memory 44b. As further described below, the patient-controllable circuitry allows the wearer of the garment 100 to self-treat the condition from which he suffers by allowing him to self-adjust the heat output to any and all muscles, individually or in combination, and similarly to self-adjust the amount of electrical stimulation provided to the muscles.

The above-described patient-treatment system may also include a housing (e.g., a belt pack) 40 that features an antenna 45 for wireless communication and a USB port 46, and may be structured and arranged to accommodate a battery 48, the CPU 42, and memory 44. The housing may be attachable to, for example, a waistline of the wearable garment 100, a waistline of another item of clothing worn by the patient, or a belt 49 worn by the patient. In such a fashion, the patient may employ a handheld device 50 to wirelessly transmit heating instructions to the CPU 42. For example, the patient may adjust the temperature of the heat output by a heating element 15, control a first heating element in a first panel 47a independently of a second heating element in a second panel 47b, etc., as some muscles may be more symptomatic than others. The patient-provided instructions and any data recorded from the patient's body (using, for example, the afore-described lead 11 and/or thermal sensor 17) may also be stored in the computer memory 44.

Advantageously, the CPU 42 may be adapted or may include software with executable instructions to prompt the patient to accept an automatic scheduling of a therapeutic regimen based on information learned from the instructions input by the patient. For example, if the patient turns up the temperature on his left bicep every afternoon for a week, the CPU 42 may offer to store that setting for the patient and may thereafter automatically turn up the temperature on the patient's left bicep every afternoon. In addition, a clinician or other interested individual may employ a handheld device 50 to remotely interrogate the information stored in the computer memory 44 of the patient-treatment system.

In one embodiment, the electronics for the wearable garment enable the corrective treatment of the symptoms and/or ailments associated with spasticity. For example, the electronics may measure, report, and prompt physical therapy and other regimens that will lead to strengthened muscles, improved muscle habits, reduction in contracture, and increased range of motion, all of which correct the most disabling effects of spasticity. In one embodiment, the CPU 42 executes a software program and provides a user interface for managing and controlling the measuring of the patient's condition and status, as well as for reporting to, prompting, and alerting both the patient and clinicians. In this way, the electronics are able to measure the wearable garment's effectiveness over time. For example, the electronics may be employed to detect the improvement in the size of a muscle's electrical output (indicating strength gains), the improvement in the timing of the muscle's activity (indicating improved motor control), and the decrease in the contraction of antagonist muscles (indicating decreased spasticity). Many of these improvements depend upon regular practice both with a therapist and at home. Accordingly, in one embodiment, the electronics further measure how often the patient actually performs his therapy, thereby making very valuable data available for study, as feedback to the therapist, and for improvements in design.

The power source, e.g., battery 48, may be a lithium-ion battery, or any other energy source used now or in the future that safely delivers the energy required. The battery may have multi-level surge protection for safety, with a built-in temperature controller and LED indicator, and a low battery alarm. The garment 100 may include one or more adjustable heat setting dials, which may be independent of the garment or worn over the arm, forearm, upper or lower leg, trunk, or any convenient combination thereof. Alternatively, as described above, the heat setting dials may be adjustable via a software application.

As also mentioned, the garment 100 may include a monitor(s) 17 to measure and record skin temperature at a discrete location, i.e., panel 47, throughout the garment 100. The type of heat supplied to the muscles may be mid-, long- and/or far infrared wavelength, or any other type of heat used now or in the future, that will safely warm the intended muscles and tendons. The wiring and heating elements 15 may be made of copper, stainless steel, polymer, carbon fiber, silicone rubber, kapton, or any type of wiring used now or in the future that will most safely and efficiently conduct the energy from the battery 48 to the heating element 15. Each heating panel 47 and the heating element(s) 15 embedded therein may have custom designed and manufactured features (e.g., size, shape, temperature settings, etc.). Such custom design and manufacture may be provided for, for example, by EXO2 of Locust Grove, Ga. The heating panels 47 may have cut-outs so that other components (e.g., the afore-described thermal sensors 17, electrodes 13, and leads 11) may be integrated therewith.

A patient with spasticity in multiple extremities might require several such garments 100, each of which may be independently adjustable. In addition to treating arms and legs, the present system may be adapted to gloves, socks, or a vest of similar material if needed for an individual patient, since the muscles of the torso are known to be affected by spasticity, and by the sensation unique to Willis-Ekbom disease. The garments 100 may be designed to be independently donned and doffed by the wearer, with zippers, buttons, Velcro, or other forms of enclosure.

Each garment 100 (or portion thereof) over the extremity or trunk may have custom-fit wired patches or sections of material that cover the surface of each major muscle group and associated tendons, and each patch may be capable of heating the muscle, stimulating the muscle with electrical stimulation, surface vibration to decrease spasticity, and monitoring the muscle's inherent electrical activity during rest, stretch, normal activities of daily living, and/or exercise. In certain embodiments, individual patches or sections may be controlled individually and/or in user programmable groups.

As previously described, the patient-treatment system described herein may be controlled in part or entirely by a programmable computer-based system, including, but not limited to, Smartphone applications. The programmable computer based system may also be interfaced, e.g., wirelessly, using a USB, and the like, with one or more patient monitoring systems for controlling and reacting to certain patient readings.

The patient-treatment system may also monitor muscles that oppose the intended movement (i.e., “antagonist” muscles). For example, during both stretching and strengthening exercise, sensors may count the number of repetitions performed, and the amount of force or stretch generated, as well as the duration of each exercise. All of the data recorded through the device may be directly downloadable into another computer system through commonly used devices.

In certain embodiments, the device is capable of generating a three-dimensional stick figure representation of the patient's walking patterns, and upper extremity movement patterns, in an effort to provide biofeedback that demonstrates where the patient needs to focus, in terms of stretching or strengthening, in order to improve his movements.

The overall device applied to the individual patient, meaning any or all of the listed monitors or stimulating components, may be tailored to that individual patient's specific deficits and motor firing patterns, as determined by clinical examination, surface EMG monitoring, and/or gait analysis testing.

In another embodiment of the present invention, heating elements 15 are incorporated into braces for the treatment of spasticity. In still another embodiment of the present invention, a heating element 15 or heat liner is added to a compressive or vibratory garment, such as those used in the treatment of Willis-Ekbom disease.

In addition to the specific heat delivery systems described above, it should be noted that other delivery systems fall within the scope of the invention. For example, embodiments of the present invention can be incorporated into articles specifically for use at a medical facility (such as a heat suit for use in a physical therapy setting), into clothing that can be worn during everyday activities, and/or into other heat delivery systems such as a blanket that can provide varying amounts of heat to different parts of the body while at rest or asleep.

The afore-described batteries 48 may be recharged through an adaptor to a standard wall socket, but can also be recharged by the transfer of energy generated by exercises performed by the patient. These exercises may or may not be recommended to help overcome that specific patient's pattern of weakness due to his disease or injury.

The battery system 48 can also power recreational toys for adults or children, which may or may not be educational, in a way that is targeted to improve the patient's function. These toys may challenge and educate the patient's cognitive or motoric pathways, and may involve accessing the internet or interacting with systems such as the Wii (manufactured by Nintendo Co., Ltd. of Kyoto, Japan), to facilitate benefit from playful exercise. For instance, simply swinging the arms or legs may substitute for moving a device such as a bat or golf club that is Wii compatible, for patients who have difficulty grasping objects. The patient-treatment system may interact with a typical motion-based video game in which the patient's movements are detected by the garment, and those movements replicated by a figure in the game, with the intention, again, of strengthening the patient's movements and coordination in a playful, bio-feedback based manner.

Other systems, methods, features, and advantages of the present invention for treating these types of disorders will be or become apparent to one with skill in the art. It is intended that all such additional systems, methods, features, and advantages be included within this description, be within the scope of the present invention, and be protected by the accompanying claims.

What is claimed is:

1. A patient-treatment system, comprising:
a wearable garment;
at least one heating element, coupled to the garment, for applying heat to a body portion of a wearer of the garment to elicit a response that diminishes symptoms of a condition suffered by the wearer; and
wearer-controllable circuitry for adjusting the heat output by the heating element, thereby allowing the wearer to self-treat the condition, the circuitry being further configured to prompt the wearer to accept an automatic scheduling of a therapeutic regimen based on information learned from instructions input by the wearer.

2. The system of claim 1, wherein the condition suffered by the wearer is at least one of spasticity, Willis-Ekbom disease, contracture, sleep onset insomnia, and sleep maintenance insomnia.

3. The system of claim 1, wherein the garment comprises inner and outer layers.

4. The system of claim 3, wherein the heating element is positioned between the inner and outer layers.

5. The system of claim 1, wherein the heating element is configured to apply the heat at far infrared wavelengths.

6. The system of claim 1 further comprising an electrode, coupled to the garment, for providing electrical stimulation to the body portion of the wearer of the garment.

7. The system of claim 1 further comprising a lead, coupled to the garment, for monitoring muscle activity in the body portion of the wearer of the garment.

8. The system of claim 1 further comprising a thermal sensor, coupled to the garment, for monitoring a skin temperature of the wearer of the garment.

9. The system of claim 1, wherein the garment comprises an insulating material that promotes retention of the heat.

10. The system of claim 1 further comprising a housing that houses at least a portion of the circuitry.

11. The system of claim 10, wherein the housing is attachable to at least one of a waistline of the wearable garment, a waistline of another item of clothing worn by the wearer of the garment, and a belt worn by the wearer of the garment.

12. The system of claim 10 further comprising a battery housed by the housing.

13. The system of claim 1, wherein the circuitry is remotely controllable by a handheld device employed by the wearer of the garment.

14. The system of claim 1 further comprising computer memory in electrical communication with the circuitry.

15. The system of claim 14, wherein the computer memory stores at least one of i) the instructions input by the wearer of the garment and ii) data recorded from the wearer's body.

16. The system of claim 14, wherein the computer memory is remotely interrogable by a clinician.

17. The system of claim 1, wherein the circuitry permits the wearer of the garment to adjust a temperature of the heat output by the heating element.

18. The system of claim 1, wherein the circuitry permits the wearer of the garment to control a first heating element independently from a second heating element.

19. A method of treating a patient, the method comprising the steps of:
applying heat to a body portion of the patient via at least one heating element coupled to a garment worn by the patient, the heat eliciting a response that diminishes symptoms of a condition suffered by the patient, the condition selected from the group consisting of spasticity, Willis-Ekbom disease, contracture, sleep onset insomnia, and sleep maintenance insomnia.

20. The method of claim 19 further comprising adjusting the applied heat in response to instructions input by the patient while self-treating the condition.

21. The method of claim 20 further comprising prompting the patient to accept an automatic scheduling of a therapeutic regimen based on information learned from the instructions input by the patient.

22. The method of claim 19, wherein the garment comprises inner and outer layers.

23. The method of claim 22, wherein the heating element is positioned between the inner and outer layers.

24. The method of claim 19, wherein the heat is applied at far infrared wavelengths.

25. The method of claim 19 further comprising electrically stimulating the body portion of the patient.

26. The method of claim 19 further comprising monitoring muscle activity in the body portion of the patient.

27. The method of claim 19 further comprising monitoring a skin temperature of the patient.

28. The method of claim 19, wherein the garment comprises an insulating material that promotes retention of the heat.

29. The method of claim 19 further comprising receiving, at a receiver associated with the garment, a wireless signal comprising instructions for controlling the application of the heat.

30. The method of claim 19 further comprising storing, in computer memory associated with the garment, at least one of i) instructions input by the patient and ii) data recorded from the patient's body.

31. The method of claim 19 further comprising receiving, at a receiver associated with the garment, a wireless signal comprising instructions to interrogate computer memory associated with the garment.

32. The method of claim 19 further comprising adjusting a temperature of the applied heat in response to instructions input by the patient.

33. The method of claim 19 further comprising controlling a first heating element independently from a second heating element.
